(19) United States
(12) Patent Application Publication
(10) Pub. No.: US 2006/0030823 A1
(43) Pub. Date: Feb. 9, 2006

(54) Auto shut off device in intravenous infusion set

(76) Inventor: Ju-Ti Wang, Taipei County (TW)

Correspondence Address: Kang De Bao Biotechnology Co., Ltd., P.O. Box No. 6-57, Junghe, Taipei, 235, TW

(21) Appl. No.: 10/912,138

(22) Filed: Aug. 6, 2004

(51) Int. Cl. A61M 5/14

(52) U.S. Cl. 604/251

(57) ABSTRACT

The present invention relates to an automatic shut off device in an intravenous infusion set having a dripping bottle, with characteristics that a circular plug formed inside, wherein, a silicon film formed inside the circular plug, a thick rim formed on a perimeter of the silicon film, and a cylinder pipe formed on a bottom of the dripping bottle to connect on a bottom with a tube. With a structure hereinbefore, air can be prevented from entering an individual body through the tube, and also a retro-flow of blood can be prevented, thereby enhancing safety in medical treatment.

### BACKGROUND OF THE INVENTION

(a) Field of the Invention

The present invention relates to an automatic shut off device in an intravenous infusion set, which prevents air from entering an individual body through a tube and also prevents a retro-flow of blood, thereby enhancing safety in medical treatment.

(b) Description of the Prior Art

Referring to FIG. 5. A conventional intravenous infusion set includes a medicament solution bottle 7 and an infusion set 8, having a needle 81, a dripping bottle 80, a tube 82, a flow-rate adjustment roller 83, and a hypodermic needle 84. Solution in the medicament solution bottle 7 flows through the needle 81, the dripping bottle 80, the tube 82, the flow-rate adjustment roller 83, and the hypodermic needle 84 into an individual body. By adjusting the flow-rate adjustment roller 83, a desired solution flow rate can be achieved.

The conventional intravenous infusion set works well until solution flowing from the medicament solution bottle 7 to the dripping bottle 80 exhausts, when air in the tube 82 follows to enter the individual body, thereby forming air embolism in blood, which causes heart failure, brain embolism or lung embolism. As a result, serious coma or even death may follow.

### SUMMARY OF THE INVENTION

The present invention relates to an automatic shut off device in an intravenous infusion set comprising a needle formed atop a dripping bottle, with characteristics that a circular plug formed inside thereof, wherein a silicon film with a thick rim formed inside, and a cylinder pipe formed on a bottom of the dripping bottle, to be connected on a bottom with a tube.

When solution level in the dripping bottle is low enough, the circular plug will be falling down till the silicon film seals the cylinder pipe, thereby preventing air from entering the tube through the cylinder pipe and enhancing safety in medical treatment.

To enable a further understanding of the said objectives and the technological methods of the invention herein, the brief description of the drawings below is followed by the detailed description of the preferred embodiments.

### BRIEF DESCRIPTION OF THE DRAWING

FIG. 1 shows an exploded elevational view of the present invention.

FIG. 2 shows a perspective view of a shut off device in a dripping bottle of the present invention.

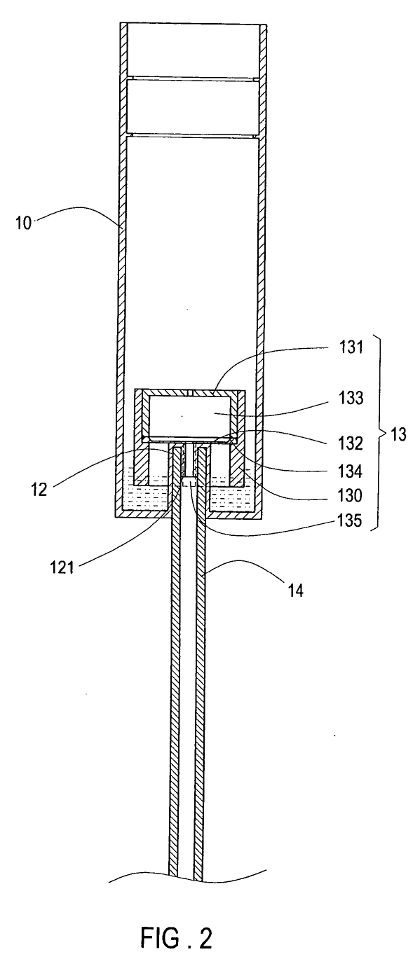

FIG. 3 shows the operation mechanism of the present invention.

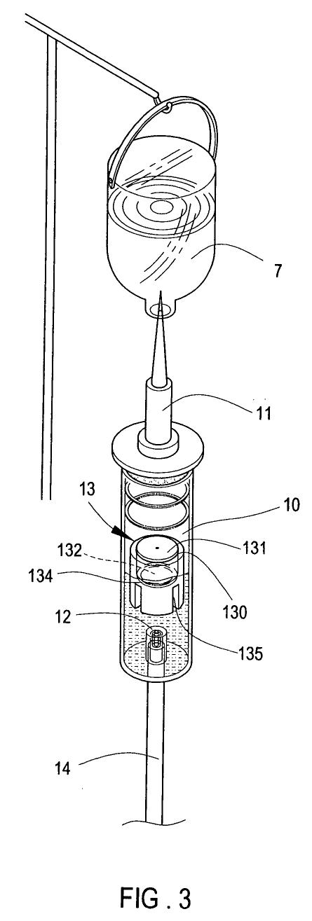

FIG. 4 shows the operation mechanism of the present invention.

FIG. 5 shows a perspective view of a conventional infusion set.

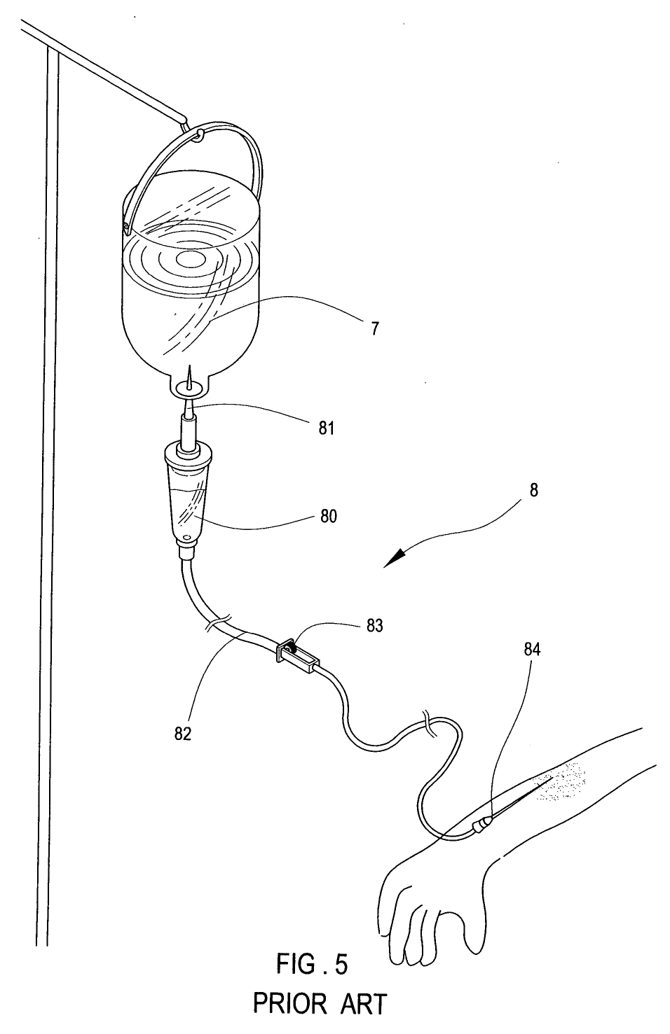

### DETAILED DESCRIPTION OF THE PREFERRED EMBODIMENTS

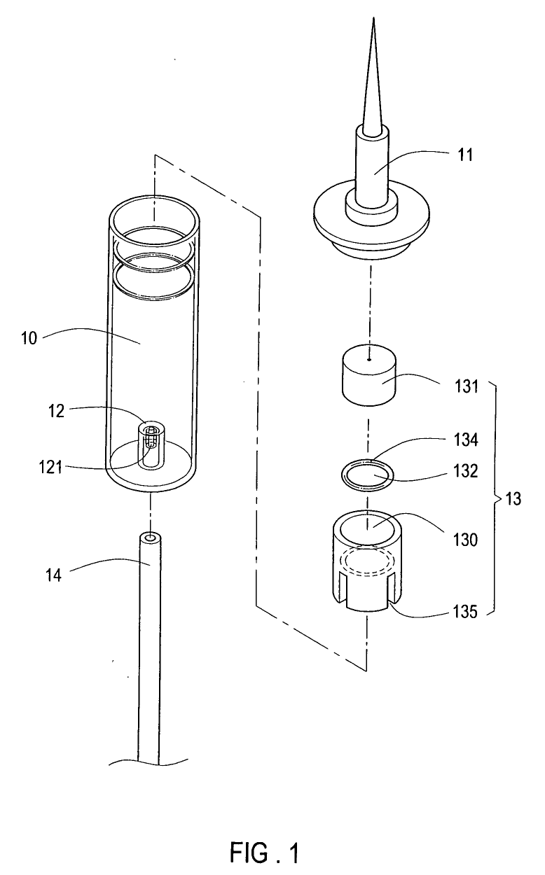

Referring to FIG. 1˜3. The present invention relates to an automatic shut off device in an intravenous infusion set having a needle 11 formed atop a dripping bottle 10, with characteristics that a cylinder pipe 12 formed on a bottom and a circular plug 13 formed inside, wherein, a sleeve 130 covering a perimeter of a lid 131 from a top, grooves 135 formed 90° apart on a lower section of a perimeter of the sleeve 130, a silicon film 132 formed in the lid 131, a thick rim 134 formed on a perimeter of the silicon film 132 positioning between the sleeve 130 and the lid 131, an air chamber 133 formed between the lid 131 and the silicon film 132, and a top of the cylinder pipe 12 curved downwards to form a connection section 121 to connect with a tube 14 perforating through a bottom of the dripping bottle 10.

Stabbing the needle 11 to a medicament solution bottle 7 will cause solution thereof flows to the dripping bottle 10. With a reserved buoyancy of the air chamber 133 and when solution level in the dripping bottle 7 is high enough, the circular plug 13 floats, allowing air below the circular plug 13 escapes from grooves 135, thereby enabling solution to stream through the cylinder pipe 12 to the tube 14.

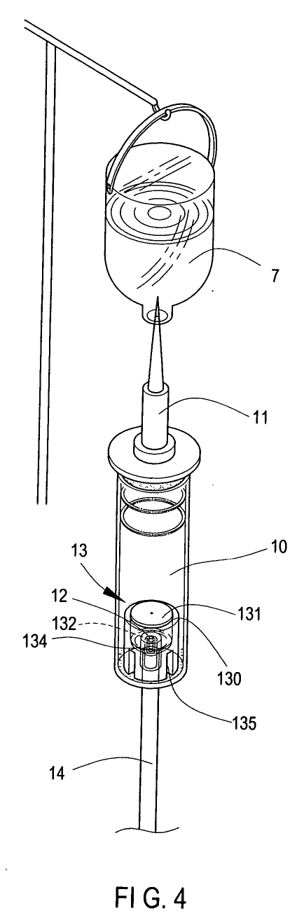

Referring to FIG. 4. When solution in the medicament solution bottle 7 is dripping to the end, the floating circular plug 13 will be falling accordingly and the silicon film 132 of the circular plug 13 will be finally falling on the cylinder pipe 12 when solution level falling below the top of the cylinder pipe 12. With characteristics of elasticity and extensibility, the silicon film 132 prevents air from entering the tube 14 through the cylinder pipe 12 and also prevents a retro-flow of blood, thereby enhancing safety in medical treatment.

It is of course to be understood that the embodiment described herein is merely illustrative of the principles of the invention and that a wide variety of modifications thereto may be effected by persons skilled in the art without departing from the spirit and scope of the invention as set forth in the following claims.

What is claimed is:

1. An automatic shut off device in an intravenous infusion set comprising a needle formed atop a dripping bottle, with characteristics that a cylinder pipe formed on a bottom and a circular plug formed inside, wherein, a sleeve covering a perimeter of a lid from a top, grooves formed 90° apart on a lower section of a perimeter of the sleeve, a silicon film formed in the lid, a thick rim formed on a perimeter of the silicon film positioning between the sleeve and the lid, an air chamber formed between the lid and the silicon film, and a top of the cylinder pipe curved downwards to form a connection section to connect with a tube perforating through the bottom of the dripping bottle.
